(19) United States
(12) Patent Application Publication
(10) Pub. No.: US 2013/0324829 A1
(43) Pub. Date: Dec. 5, 2013

(54) Adjustable Suture

(71) Applicants: Queen's University at Kingston, (US); Hotel Dieu Hospital, (US)

(72) Inventors: Kong Ying Xie, Kingston (CA); Robert James Campbell, Kingston (CA); Yong Jun Lai, Kingston (CA)

(73) Assignees: Hotel Dieu Hospital, Kingston (CA); Queen's University at Kingston, Kingston (CA)

(21) Appl. No.: 13/684,346

(22) Filed: Nov. 23, 2012

Related U.S. Application Data
(60) Provisional application No. 61/563,707, filed on Nov. 25, 2011.

(51) Int. Cl. A61B 17/06; A61B 3/16

(52) U.S. Cl. CPC A61B 17/06166; A61B 3/16
USPC 600/398; 606/228; 156/73.2

(57) ABSTRACT

Provided are adjustable sutures comprising a suture thread 1 attached to one or more tension-releasing portion(s) 2, wherein the suture thread 1 and the tension-releasing portion(s) 2 are joined together at two or more joining zones 3, and a spanning segment 4 extends between the joining zones 3, and wherein the adjustable suture is breakable, mechanically or by applying laser energy. The adjustable sutures may be used in surgical methods where intra-operative and/or post-operative adjustment of the suture is required.

### RELATED APPLICATIONS

This application claims the benefit of the filing date of U.S. Provisional Patent Application No. 61/563,707, filed on Nov. 25, 2011, the contents of which are incorporated herein by reference in their entirety.

### FIELD OF THE INVENTION

This invention relates to adjustable sutures for use in surgical methods. The adjustable sutures may be used in surgical methods where intra-operative and/or post-operative adjustment of the suture is required, such as trabeculectomy for treating glaucoma.

### BACKGROUND OF THE INVENTION

Glaucoma is a degenerative disease of the optic nerve and is the leading cause of irreversible blindness in the world (Dimitrov, P. N. et al., Invest. Ophthalmol. Vis. Sci., 2003, 44: 5075-81; Resnikoff, S. et al., Bull World Health Organ., 2004, 82: 844-51). While a number of risk factors for this disease have been identified, the most important modifiable risk factor is elevated pressure within the eye, termed intraocular pressure (IOP). The internal structures of the eye are bathed in a constantly secreted fluid known as the aqueous humour. This fluid serves functions carried out by the blood system in most parts of the body, including delivery of oxygen and vital chemicals, and the removal of metabolic products. Aqueous humour is secreted into the eye by the ciliary body and must subsequently exit the eye via one of two routes: through the trabecular meshwork (a complex sieve-like structure), or through the uveal structures (iris and ciliary body). As a consequence of many pathologic processes, this outflow system may be compromised with a resultant increase in outflow resistance, causing the intraocular pressure to rise.

Current therapies for glaucoma are directed at lowering IOP. While medical treatments are generally the first line therapies, a large percentage of patients require surgical intervention to prevent blindness (Campbell, R. J. et al., Canadian Journal of Ophthalmology A, 2008, 43(4): 449-53; Ramulu, P. Y. et al., Ophthalmology, 2007, 114(12): 2265-70). In 2004, over 6,000 glaucoma operations were performed in Canada, and over 40,000 in the USA (Campbell, R. J. et al., Canadian Journal of Ophthalmology A, 2008, 43(4), pp. 449-53; Ramulu, P. Y. et al., Ophthalmology, 2007, 114(12), pp. 2265-70). Failure of these surgeries and consequent inadequate IOP reduction lead to progressive optic nerve degeneration and blindness. Blindness has a tremendously negative effect on quality of life, which is equal to, or greater than that caused by a major stroke (Post, P. N. et al., Stroke, 2001, 32(6): 1425-9). Moreover, the estimated cost of blindness in Canada is 7.9 billion dollars annually (Buhrmann, R. et al., Foundations for a Canadian Vision Health Strategy, Toronto: The National Coalition for Vision Health, 2007).

The most common surgical approach to treating glaucoma is termed “trabeculectomy.” Trabeculectomy involves the creation of a new low resistance fluid outflow pathway by fashioning a small opening (fistula) between the inside of the eye (anterior chamber) and the “sub-conjunctival” space (Jones, E. et al., Curr. Opin. Ophthalmol., 2005, 16: 107-13). In order to prevent the eye pressure from dropping too low, which may lead to complications and/or blindness, a scleral flap (a guarding trap-door of tissue) is created over the fistula. This scleral flap has traditionally been secured via standard sutures, which are left slightly loose to allow low-resistance egress of the fluid from the eye. The aqueous humour is then captured under the mucus membrane layer of the eye called the conjunctiva, and then reabsorbed by the body.

Unfortunately, with current treatment procedures trabeculectomy surgery is risky. The required suture tension to attain the desired pressure lowering within the eye is difficult to gauge precisely; this difficulty is of particular concern since vision threatening complications may result if the pressure in the eye is lowered too far (Geddea, S. J. et al., American Journal of Ophthalmology, 2009, 148(5):670-84). Hence, to avoid overly low pressures, sutures are generally tied somewhat tight during initial surgery with a plan to release the sutures post-operatively. Because laser energy can cross the thin conjunctiva overlaying the scleral flap sutures, a laser is commonly used to break the sutures as needed in the post-operative period. However this breakage of sutures is frequently associated with a precipitous drop in eye pressure, which may cause serious problems including severe hemorrhage within the eye and loss of vision.

Research and development in the field of adjustable sutures for glaucoma surgery has been very limited. Using present surgical techniques, surgeons may completely release sutures with a laser. This is an all-or-none procedure, and thus often leads to serious complications as a result of a precipitous drop in IOP. A second technique involves tying sutures that are not knotted. In some patients these may be loosened mechanically in the postoperative phase. However, this is risky and fails to work in the majority of cases because the suture cannot be grasped mechanically and fails to loosen. This latter technique also has the disadvantage of potentially leading to serious complication because when the suture is grabbed mechanically, serious damage to the overlying structure, the conjunctiva, may occur. Laser adjustable suture techniques and designs have been reported, but they are generally impractical, uneconomical, ineffective, or unable to work in an incremental fashion, and have not gained wide clinical acceptance (see, e.g., U.S. Pat. No. 5,651,377; Wells, A. P. et al., J Glaucoma, 2004, 13:400-6; Davis, A. P., Ophthalmic surgery, lasers & imaging, 2006, 37(3):252-56). Current surgical approaches remain unable to precisely regulate and titrate postoperative IOP.

There is a need therefore for easy-to-use, reliable and/or cost-effective tools and methods for controlling IOP after trabeculectomy surgery and/or for improving glaucoma surgical success rates.

### SUMMARY OF THE INVENTION

In a first aspect, the invention provides an adjustable suture comprising a suture thread and a tension-releasing portion, wherein the tension-releasing portion is adapted to accept tension when the adjustable suture is cut at a selected site. In an embodiment of this aspect wherein the suture thread comprises a portion which is a spanning segment, and the selected site is on the spanning segment of the adjustable suture or at a junction between the spanning segment and the tension-releasing portion. In an embodiment of this aspect, the selected site is not on the tension-releasing portion. In another embodiment of this aspect, the tension-releasing portion is not aligned with the longitudinal axis of the suture thread. In an embodiment of this aspect, the tension-releasing portion is fixed in place on the adjustable suture. In yet another embodiment of this aspect, the tension-releasing portion is integrated with the suture thread. In an embodiment of this aspect, the adjustable suture is lengthened or loosened when the adjustable suture is cut at the selected site. In another embodiment of this aspect, the adjustable suture comprises more than one tension-releasing portion. In an embodiment of this aspect, each tension-releasing portion is independently releasable. In another embodiment of this aspect, the adjustable suture is further lengthened or loosened, after a first tension-releasing portion has been cut, by cutting a second tension-releasing portion. In yet another embodiment of this aspect, the second tension-releasing portion is adapted to release tension when the adjustable suture is cut at a second selected site. In an embodiment of this aspect, the second selected site is on at least one spanning segment or at a junction between the at least one spanning segment and the second tension-releasing portion. In an embodiment of this aspect, the second selected site is on the second tension-releasing portion. In another embodiment of this aspect, the adjustable suture is cut by breaking the adjustable suture mechanically or by applying laser energy to the adjustable suture. In an embodiment of this aspect, the tension-releasing portion is substantially ring-shaped, rectangular-shaped or triangular-shaped. In another embodiment of this aspect, the adjustable suture is substantially φ-, D-, Δ-, $-, B-, 8- or §-shaped. In an embodiment of this aspect, the adjustable suture comprises nylon, silicone elastic polymer, Silastic®, silicone rubber, silk, polyester, polypropylene and/or other biocompatible suture materials. In an embodiment of this aspect, the adjustable suture comprises nylon 6 or nylon 6.6. In an embodiment of this aspect, the nylon's caliber is 2-0 nylon, 3-0 nylon, 5-0 nylon, 6-0 nylon, 8-0 nylon or 10-0 nylon. In yet another embodiment of this aspect, the adjustable suture further comprises nylon adhesive. In an embodiment of this aspect, the suture thread and the tension-releasing portion are made of the same material. In an embodiment of this aspect, the suture thread and the tension-releasing portion are made of different materials. In an embodiment of this aspect, the adjustable suture is a laser-adjustable suture. In another embodiment of this aspect, the suture thread is made of material breakable by applying laser energy, and the tension-releasing portion is made of material which is not breakable by applying laser energy. In an embodiment of this aspect, the tension-releasing portion is made of deformable or flexible material. In an embodiment of this aspect, the tension-releasing portion comprises nylon 6, nylon 6.6 or silicone elastic polymer. In yet another embodiment of this aspect, the tension-releasing portion is substantially ring-shaped. In an embodiment of this aspect, the diameter of the tension-releasing portion is about 0.25 mm to about 1.5 mm. In an embodiment of this aspect, the adjustable suture comprises spin coated nylon, silicone elastic polymer, Silastic®, silicone rubber, silk, polyester, polypropylene, biocompatible suture materials, or any combination thereof. In an embodiment of this aspect, different portions of the adjustable suture are visually distinguishable from one another, by, for example, use of different colours and/or textures.

In a second aspect, the invention provides an adjustable suture comprising a suture thread attached to one or more tension-releasing portion(s), wherein the suture thread and the tension-releasing portion are joined together at two or more joining zones, and one or more spanning segment(s) extend between the joining zones, wherein the adjustable suture is breakable mechanically or by applying laser energy, and wherein, when more than one tension-releasing portion is present, then more than one spanning segment is also present, and each of said spanning segments extending between a pair of adjacent joining zones is independently breakable. In an embodiment of this aspect, the adjustable suture is lengthened or loosened each time one of the at least one spanning segments is broken. In another embodiment of this aspect, the adjustable suture comprises one tension-releasing portion. In an embodiment of this aspect, the adjustable suture comprises more than one tension-releasing portion. In yet another embodiment of this aspect, the adjustable suture is lengthened or loosened when at least one of said tension-releasing portion(s) is broken, as long as at least one of the spanning segments has previously been broken. In an embodiment of this aspect, at least one tension-releasing portion(s) is substantially ring-shaped, rectangular-shaped or triangular-shaped. In an embodiment of this aspect, the adjustable suture is substantially φ-, D-, Δ-, $-, B-, 8- or §-shaped. In another embodiment of this aspect, the adjustable suture comprises nylon, silicone elastic polymer, Silastic®, silicone rubber, silk, polyester, polypropylene and/or other biocompatible suture materials. In an embodiment of this aspect, the adjustable suture comprises nylon 6 or nylon 6.6. In an embodiment of this aspect, the caliber of the nylon is 2-0 nylon, 3-0 nylon, 5-0 nylon, 6-0 nylon, 8-0 nylon or 10-0 nylon. In another embodiment of this aspect, the adjustable suture further comprises nylon adhesive. In an embodiment of this aspect, the suture thread and the one or more tension-releasing portion(s) are made of the same material. In an embodiment of this aspect, the suture thread and the one or more tension-releasing portion(s) are made of different materials. In another embodiment of this aspect, the adjustable suture is a laser-adjustable suture. In an embodiment of this aspect, the suture thread is made of material breakable by applying laser energy, and the tension-releasing portion is made of material which is not breakable by applying laser energy. In yet another embodiment of this aspect, the one or more tension-releasing portion(s) are made of deformable or flexible material. In an embodiment of this aspect, the one or more tension-releasing portion(s) comprise nylon 6, nylon 6.6 or silicone elastic polymer. In another embodiment of this aspect, the one or more tension-releasing portion(s) is substantially ring-shaped. In an embodiment of this aspect, the diameter of at least one of the tension-releasing portion(s) is about 0.25 mm to about 1.5 mm.

In a third aspect, the invention provides a method of treating glaucoma in a subject in need thereof, comprising a) performing trabeculectomy using the adjustable suture defined in any one of claims 29 to 47, b) monitoring the subject's intraocular pressure (IOP) post-operatively, and c) if lower IOP is desired, breaking at least one spanning segment, such that the adjustable suture is loosened and IOP is lowered. In an embodiment of this aspect, the spanning segment is broken by applying laser energy. In an embodiment of this aspect, the method further comprises repeating steps b) and c) until the IOP in the subject is lowered to a selected level. In another embodiment of this aspect, when steps b) and c) are repeated, either another spanning segment is broken or at least one of the tension-releasing portion(s) is broken. In an embodiment of this aspect, the tension-releasing portion is broken by applying laser energy. In another embodiment of this aspect, steps b) and c) are repeated until the IOP in the subject is about 5 mm Hg to about 15 mm Hg. In an embodiment of this aspect, breaking the spanning segment or the tension-releasing portion lowers the IOP by about 1 to about 5 mm Hg, about 5 mm Hg to about 10 mm Hg, about 10 mm Hg to about 20 mm Hg, about 10 mm Hg to about 30 mm Hg, about 1 mm Hg, about 3 mm Hg, about 5 mm Hg, about 7 mm Hg, about 10 mm Hg, about 15 mm Hg, about 20 mm Hg, about 25 mm Hg or about 30 mm Hg in the subject. In another embodiment of this aspect, breaking the spanning segment or the tension-releasing portion lowers the IOP by about 1% to about 5%, about 5%, about 10%, about 15%, about 20%, about 25%, about 30%, about 40%, about 50%, about 60%, about 70% or about 80% in the subject.

In a fourth aspect, the invention provides a method of incrementally lowering IOP post-operatively in a subject in need thereof, wherein the subject has undergone trabeculectomy surgery using the adjustable suture of the first or second aspect, comprising a) breaking at least one spanning segment by applying laser energy, such that the adjustable suture is loosened and IOP is lowered, and b) repeating step a) until the IOP in the subject is lowered to a selected level. In an embodiment of this aspect, in step b), IOP in the subject is lowered by breaking either at least one more spanning segment or at least one tension-releasing portion(s) or both.

In a fifth aspect, the invention provides a method of performing trabeculectomy in a subject in need thereof, comprising a) performing trabeculectomy using the adjustable suture defined in any one of claims 29 to 47, b) monitoring the subject's IOP post-operatively, and c) if lower IOP is desired, breaking at least one spanning segment, such that the adjustable suture is loosened and IOP is lowered and glaucoma is treated. In an embodiment of this aspect, the at least one spanning segment is broken by applying laser energy. An embodiment of this aspect, further comprises repeating steps b) and c) as needed to treat glaucoma. In an embodiment of this aspect, when steps b) and c) are repeated, either at least one more spanning segment is broken or at least one tension-releasing portion(s) is broken or both. In an embodiment of this aspect, the tension-releasing portion is broken by applying laser energy. In another embodiment of this aspect, steps b) and c) are repeated until the IOP in the subject is about 5 mm Hg to about 15 mm Hg. In an embodiment of this aspect, steps b) and c) are repeated until IOP is lowered by about 1 to about 5 mm Hg, about 5 mm Hg to about 10 mm Hg, about 10 mm Hg to about 20 mm Hg, about 10 mm Hg to about 30 mm Hg, about 1 mm Hg, about 3 mm Hg, about 5 mm Hg, about 7 mm Hg, about 10 mm Hg, about 15 mm Hg, about 20 mm Hg, about 25 mm Hg or about 30 mm Hg in the subject. In another embodiment of this aspect, steps b) and c) are repeated until IOP is lowered by about 1% to about 5%, about 5%, about 10%, about 15%, about 20%, about 25%, about 30%, about 40%, about 50%, about 60%, about 70% or about 80% in the subject. In an embodiment of this aspect, breaking the spanning segment or the tension-releasing portion lowers the IOP by about 1 to about 5 mm Hg, about 5 mm Hg to about 10 mm Hg, about 10 mm Hg to about 20 mm Hg, about 10 mm Hg to about 30 mm Hg, about 1 mm Hg, about 3 mm Hg, about 5 mm Hg, about 7 mm Hg, about 10 mm Hg, about 15 mm Hg, about 20 mm Hg, about 25 mm Hg or about 30 mm Hg in the subject. In an embodiment of this aspect, breaking the spanning segment or the tension-releasing portion lowers the IOP by about 1% to about 5%, about 5%, about 10%, about 15%, about 20%, about 25%, about 30%, about 40%, about 50%, about 60%, about 70% or about 80% in the subject.

In a sixth aspect, the invention provides a method of treating glaucoma in a subject in need thereof, comprising a) performing trabeculectomy using the adjustable suture defined in any one of claims 29 to 47, b) monitoring the subject's IOP post-operatively, c) if lower IOP is desired, breaking at least one spanning segment 4, such that the adjustable suture is loosened and IOP is lowered, d) monitoring again the subject's IOP post-operatively, e) if lower IOP is desired, breaking either another spanning segment 4 or at least one tension-releasing portion(s), and f) optionally repeating steps d) and e) until IOP in the subject is lowered to a selected level or glaucoma is treated in the subject.

In a seventh aspect, the invention provides a laser-adjustable suture comprising a suture thread attached to one or more tension-releasing portion(s), wherein the suture thread and the tension-releasing portion are joined together at two or more joining zones, and a spanning segment of the suture extends between the joining zones, wherein the laser-adjustable suture is breakable by applying laser energy, and wherein, when more than one tension-releasing portion is present, more than one spanning segment is present, each of said spanning segments extending between a pair of adjacent joining zones and independently breakable by applying laser energy.

In an eighth aspect, the invention provides a method of treating glaucoma in a subject in need thereof, comprising a) performing trabeculectomy using the adjustable suture defined in any one of claims 1 to 28, b) monitoring the subject's IOP post-operatively, and c) if lower IOP is desired, cutting the adjustable suture at the selected site to release tension, such that the adjustable suture is lengthened or loosened. In an embodiment of this aspect, IOP is lowered in the subject when the adjustable suture is lengthened or loosened. In an embodiment of this aspect, the adjustable suture comprises more than one tension-releasing portion. An embodiment of this aspect further comprises steps of d) monitoring again the subject's IOP post-operatively, e) if lower IOP is desired, cutting the adjustable suture at a second selected site, and f) optionally repeating steps d) and e) until IOP in the subject is lowered to a selected level or glaucoma is treated in the subject. In an embodiment of this aspect, the adjustable suture is lengthened or loosened each time it is cut. In an embodiment of this aspect, lengthening or loosening the adjustable suture lowers IOP in the subject. In an embodiment of this aspect, the adjustable suture is cut by applying laser energy. In an embodiment of this aspect, the selected site is on a spanning segment or at a junction between the spanning segment and the tension-releasing portion. In an embodiment of this aspect, the selected site is not on the tension-releasing portion. In an embodiment of this aspect, the second selected site is on at least one spanning segment, at a junction between the at least one spanning segment and the tension-releasing portion, or on the tension-releasing portion. In yet another embodiment of this aspect, steps d) to f) are repeated until the IOP in the subject is about 5 mm Hg to about 15 mm Hg. In an embodiment of this aspect, cutting the laser adjustable suture lowers the IOP in the subject by about 1 to about 5 mm Hg, about 5 mm Hg to about 10 mm Hg, about 10 mm Hg to about 20 mm Hg, about 10 mm Hg to about 30 mm Hg, about 1 mm Hg, about 3 mm Hg, about 5 mm Hg, about 7 mm Hg, about 10 mm Hg, about 15 mm Hg, about 20 mm Hg, about 25 mm Hg or about 30 mm Hg. In an embodiment of this aspect, cutting the laser adjustable suture lowers the IOP in the subject by about 1% to about 5%, about 5%, about 10%, about 15%, about 20%, about 25%, about 30%, about 40%, about 50%, about 60%, about 70% or about 80%.

In a ninth aspect, the invention provides a method of incrementally lowering IOP post-operatively in a subject in need thereof, wherein the subject has undergone trabeculectomy surgery using the adjustable suture defined in any embodiment of the first aspect, comprising a) cutting the adjustable suture at the selected site such that the adjustable suture is lengthened or loosened and IOP is lowered, and b) repeating step a) until the IOP in the subject is lowered to a selected level. In an embodiment of this aspect, the adjustable suture is cut by applying laser energy. In another embodiment of this aspect, the selected level is about 5 mm Hg to about 15 mm Hg. In certain embodiments of this aspect, glaucoma is treated in the subject.

In a tenth aspect, the invention provides a method of performing trabeculectomy in a subject in need thereof, comprising a) performing trabeculectomy using the adjustable suture defined in any one of claims 1 to 28, b) monitoring the subject's IOP post-operatively, and c) if lower IOP is desired, cutting the adjustable suture at the selected site such that the adjustable suture is lengthened or loosened and IOP is lowered. In an embodiment of this aspect, glaucoma is treated in the subject.

In an eleventh aspect, the invention provides a method of treating glaucoma in a subject in need thereof, comprising a) performing trabeculectomy using the adjustable suture defined in any one of claims 1 to 28, b) monitoring the subject's IOP post-operatively, c) if lower IOP is desired, cutting the adjustable suture at the selected site to release tension, such that the adjustable suture is lengthened or loosened and IOP is lowered, d) monitoring again the subject's IOP post-operatively, e) if lower IOP is desired, cutting the adjustable suture at a second selected site to release tension further, wherein the second selected site is on at least one spanning segment, at a junction between the at least one spanning segment and the tension-releasing portion, or on the tension-releasing portion, and f) optionally repeating steps d) and e) until IOP in the subject is lowered to a selected level or glaucoma is treated in the subject. In an embodiment of this aspect, the adjustable suture is cut by applying laser energy.

In a twelfth aspect, the invention provides a method of making the adjustable suture of any of the embodiments of the first aspect, comprising using a micromolding, hot embossing, ultrasonic welding, riveting, knotting, clamping, gluing, chemical welding technique or any combination thereof to form an adjustable suture. In an embodiment of this aspect, the adjustable suture comprises spin coated nylon, silicone elastic polymer, Silastic®, silicone rubber, silk, polyester, polypropylene, biocompatible suture materials, or any combination thereof.

In another aspect, the invention provides an adjustable suture comprising a suture thread and a tension-releasing portion, wherein the tension-releasing portion is adapted to release tension in the adjustable suture when the adjustable suture is cut at a selected site.

Hence, there is provided herein a surgical suture that may be lengthened or loosened without the need to remove or “re-tie” a knot. Further, the suture may be adjusted intra-operatively or post-operatively, either mechanically or by the application of laser energy. An adjustable suture as provided herein may be used in any type of surgery where intra-operative and/or post-operative adjustment of the suture is desired. For example, use of an adjustable suture is described herein with respect to trabeculactomy procedures. However, use of adjustable sutures as described herein is not limited thereto.

According to an aspect of the invention, there are provided herein adjustable sutures which allow safe, incremental intra-operative and/or post-operative adjustment suture tension. For example, adjustable sutures as provided herein may be used in trabeculectomy procedures for treatment of glaucoma, permitting adjustment of eye pressure in the post-trabeculectomy surgery period. These adjustable sutures can be adjusted using a safe laser, which has been used for decades in eye care and is widely available in ophthalmology departments, or mechanically, using for example scissors or a blade. Adjustable sutures mitigate the risk created by the inability to titrate precisely the amount of pressure lowering obtained using current glaucoma surgical techniques. Adjustable sutures may also be adjusted intra-operatively, i.e., during surgery.

According to another aspect of the invention, there is provided an adjustable suture 10 comprising a suture thread 1 and a tension-releasing portion 2, wherein a segment of the suture thread 1 spans the tension-releasing portion 2 (this segment of the suture thread 1 is referred to as the spanning segment 4), and the tension-releasing portion 2 is adapted to release tension when the adjustable suture 10 is cut at a selected site. The selected site may be, for example, on the spanning segment 4 or at a junction between the spanning segment 4 and the tension-releasing portion 2. In one embodiment, the selected site is not on the tension-releasing portion 2.

The tension-releasing portion 2 has two states. In its first state, the tension-releasing portion 2 is not tension-bearing and the spanning segment 4 bears tension. In the second state of the tension-releasing portion 2, the selected site (e.g., in the spanning segment 4 at a junction between the spanning segment 4 and the tension-releasing portion 2) has been cut such that the tension-releasing portion 2 has been released from inactivity and becomes engaged or operative, i.e., tension-bearing.

In an embodiment, the tension-releasing portion 2 is not aligned with the longitudinal axis of the suture thread 1. In another embodiment, the tension-releasing portion 2 is fixed in place on the suture thread 1. In some embodiments, the tension-releasing portion 2 is integral (e.g., fabricated as one unit) with the suture thread 1

Adjustable sutures provided herein are lengthened or loosened when cut at a selected site. Generally, cutting the adjustable suture 10 at a selected site engages (i.e., renders operative) the tension-releasing portion 2, which has an effect of lengthening or loosening the adjustable suture 10. In some embodiments, adjustable sutures comprise more than one tension-releasing portion 2. In some embodiments, when more than one tension-releasing portion 2 is present, each tension-releasing portion 2 is independently able to become operative.

In some embodiments, following cutting of the spanning segment 4, it is possible to further loosen the adjustable suture by cutting one or more of a plurality of tension-releasing portions 2. See, for example, the adjustable suture schematic of FIG. 3G. Thus, a first tension-releasing portion 2 effectively functions as a spanning segment 4 for a second tension-releasing portion 2, a second tension-releasing portion 2 functions as a spanning segment 4 for a third tension-releasing portion 2, etc. That is, when a plurality of tension-releasing portions are present, an adjustable suture is further lengthened or loosened, after a first tension-releasing portion 2 has already been cut, by cutting a second tension-releasing portion 2. This can be done by cutting an adjustable suture at a second selected site, subsequent to cutting a first selected site. These steps can be repeated as many times as permitted by the structure and accessibility of an adjustable suture until an adjustable suture has a desired tightness or looseness. When cutting an adjustable suture for a second, third, or fourth, etc., time, the adjustable suture may be cut on a spanning segment 4, at a junction between a spanning segment 4 and a tension-releasing portion 2, or on a tension-releasing portion 2.

Adjustable sutures may be cut by breaking the adjustable suture mechanically, for example using scissors or a blade, or by applying laser energy to the adjustable suture.

In some embodiments, a tension-releasing portion 2 of an adjustable suture is substantially ring-shaped, rectangular-shaped or triangular-shaped. In other embodiments, adjustable sutures are substantially φ-, D-, Δ-, $-, B-, 8- or §-shaped. In an embodiment, a tension-releasing portion 2 is substantially ring-shaped. The diameter of a tension-releasing portion may be, for example, about 0.25 mm to about 1.5 mm.

In an embodiment, an adjustable suture comprises nylon, silicone elastic polymer, Silastic®, silicone rubber, silk, polyester, polypropylene and/or other biocompatible suture materials, or combinations thereof. In another embodiment, an adjustable suture comprises nylon 6 or nylon 6.6. The caliber of nylon used to make adjustable sutures may be, for example, 2-0 nylon, 3-0 nylon, 5-0 nylon, 6-0 nylon, 8-0 nylon or 10-0 nylon. In some embodiments, adjustable sutures further comprise nylon adhesive.

In an embodiment, an adjustable suture 10 comprises a suture thread 1 and a tension-releasing portion 2, wherein the suture thread and the tension-releasing portion are made of the same material. In another embodiment, the suture thread 1 and tension-releasing portion 2 are made of different materials. When more than one tension-releasing portion 2 is present in an adjustable suture, the tension-releasing portions 2 may all be made of the same material, may all be made of a different material(s), or some of the tension-releasing portions 2 may be made of the same material while others are made of a different material(s). Similarly, some tension-releasing portions 2 may be made of the same material as the suture thread 1, whereas others are made of a different material(s). In a particular embodiment, an adjustable suture 10 comprises a suture thread 1 made of material breakable by applying laser energy, and a tension-releasing portion 2 made of material which is not breakable by applying laser energy.

In further embodiments, tension-releasing portions 2 are made of deformable or flexible material.

According to another aspect of the invention, there is provided an adjustable suture comprising a suture thread 1 attached to one or more tension-releasing portion(s) 2, wherein the suture thread 1 and the tension-releasing portion 2 are joined together at two or more joining zones 3, and one or more spanning segments 4 of the suture thread 1 extend between adjacent joining zones 3. The suture thread 1 is breakable mechanically, e.g., with scissors or a blade, or by applying laser energy. When more than one tension-releasing portion 2 is present, more than one spanning segment 4 is present, each spanning segment 4 extending between a pair of adjacent joining zones 3 and independently breakable. In an embodiment, adjustable sutures are lengthened or loosened when at least one spanning segment 4 is broken (i.e., cut). Adjustable sutures may comprise one or more than one tension-releasing portion 2. Generally, adjustable sutures are lengthened or loosened when a tension-releasing portion 2 is broken, as long as at least one spanning segment 4 has previously been broken.

According to yet another aspect of the invention, there is provided a method of treating glaucoma in a subject in need thereof, comprising: a) performing trabeculectomy using an adjustable suture; b) monitoring the subject's IOP post-operatively; and c) if lower IOP is desired, breaking at least one spanning segment 4, such that the adjustable suture is loosened or lengthened and IOP is lowered. In an embodiment, the spanning segment 4 is broken by applying laser energy. In a further embodiment, steps b) and c) are repeated until IOP in the subject is lowered to a selected level. In some embodiments, when steps b) and c) are repeated, either another spanning segment 4 is broken or a tension-releasing portion 2 is broken, e.g., by applying laser energy.

In an embodiment, steps b) and c) of the method are repeated until IOP in a subject is about 5 mm Hg to about 15 mm Hg. In another embodiment, breaking a spanning segment 4 or a tension-releasing portion 2 lowers IOP in a subject by about 1 to about 5 mm Hg, about 5 mm Hg to about 10 mm Hg, about 10 mm Hg to about 20 mm Hg, about 10 mm Hg to about 30 mm Hg, about 1 mm Hg, about 3 mm Hg, about 5 mm Hg, about 7 mm Hg, about 10 mm Hg, about 15 mm Hg, about 20 mm Hg, about 25 mm Hg or about 30 mm Hg. In yet another embodiment, breaking a spanning segment 4 or a tension-releasing portion 2 lowers IOP in a subject by about 1% to about 5%, about 5%, about 10%, about 15%, about 20%, about 25%, about 30%, about 40%, about 50%, about 60%, about 70% or about 80%.

In another embodiment, there is provided a method of incrementally lowering IOP post-operatively in a subject in need thereof, wherein the subject has undergone trabeculectomy surgery using an adjustable suture, comprising breaking a spanning segment 4, e.g., by applying laser energy, such that the adjustable suture is loosened or lengthened and IOP is lowered; and repeating this step until IOP in the subject is lowered to a selected level. IOP in the subject may be lowered by breaking either another spanning segment 4 or a tension-releasing portion 2 or both.

There are also provided methods of performing trabeculectomy in a subject in need thereof, comprising performing trabeculectomy using an adjustable suture; monitoring the subject's IOP post-operatively; and, if lower IOP is desired, breaking a spanning segment 4, such that the adjustable suture is loosened or lengthened and IOP is lowered. In an embodiment, glaucoma is treated in the subject. These steps may be repeated as needed to treat glaucoma or to lower IOP to a desired level, e.g., about 5 mm Hg to about 15 mm Hg. When the step of lowering IOP is repeated, either another spanning segment 4 is broken or a tension-releasing portion 2 is broken. In an embodiment, breaking a spanning segment 4 or a tension-releasing portion 2 lowers IOP in a subject by about 1 to about 5 mm Hg, about 5 mm Hg to about 10 mm Hg, about 10 mm Hg to about 20 mm Hg, about 10 mm Hg to about 30 mm Hg, about 1 mm Hg, about 3 mm Hg, about 5 mm Hg, about 7 mm Hg, about 10 mm Hg, about 15 mm Hg, about 20 mm Hg, about 25 mm Hg or about 30 mm Hg. In another embodiment, breaking a spanning segment 4 or a tension-releasing portion 2 lowers IOP in a subject by about 1% to about 5%, about 5%, about 10%, about 15%, about 20%, about 25%, about 30%, about 40%, about 50%, about 60%, about 70% or about 80%.

According to a further aspect of the invention, there is provided a method of treating glaucoma in a subject in need thereof, comprising: a) performing trabeculectomy using an adjustable suture; b) monitoring the subject's IOP post-operatively; c) if lower IOP is desired, breaking a spanning segment 4, such that the adjustable suture is loosened or lengthened and IOP is lowered; d) monitoring again the subject's IOP; e) if lower IOP is desired, breaking either another spanning segment 4 or one of the one or more tension-releasing portion(s) 2; and f) optionally repeating steps d) and e) until IOP in the subject is lowered to a selected level or glaucoma is treated in the subject.

In a particular embodiment, there is provided herein a laser-adjustable suture 10 comprising a suture thread 1 attached to one or more tension-releasing portion(s) 2, wherein the suture thread 1 and the tension-releasing portion 2 are joined together at two or more joining zones 3, and one or more spanning segment(s) 4 of the suture thread 1 extend between the joining zones 3; wherein the adjustable suture is breakable by applying laser energy; and wherein, when more than one tension-releasing portion is present, more than one spanning segment 4 is present, each of said spanning segments 4 extending between a pair of adjacent joining zones 3 and independently breakable by applying laser energy.

According to a further aspect of the invention, there is provided a method of treating glaucoma in a subject in need thereof, comprising performing trabeculectomy using an adjustable suture; monitoring the subject's IOP post-operatively; and, if lower IOP is desired, cutting the adjustable suture at a selected site to release tension, such that the adjustable suture is lengthened or loosened. In an embodiment, IOP is lowered in the subject when the adjustable suture is lengthened or loosened. In another embodiment, an adjustable suture comprises more than one tension-releasing portion 2. In some cases the method may further comprise steps of monitoring again the subject's IOP; if lower IOP is desired, cutting the adjustable suture at a second selected site; and optionally repeating these steps until IOP in the subject is lowered to a selected level or glaucoma is treated in the subject. In an embodiment, an adjustable suture is lengthened or loosened each time it is cut. In another embodiment, lengthening or loosening an adjustable suture lowers IOP in a subject. An adjustable suture may be cut mechanically, e.g., with scissors or a blade, or by applying laser energy.

In some embodiments, a selected site where an adjustable suture is cut is on a spanning segment 4 or at a junction between a spanning segment 4 and a tension-releasing portion 2. In another embodiment, a selected site is not on a tension-releasing portion 2. In other embodiments, when the step of cutting an adjustable suture is repeated, a second selected site is on a spanning segment 4, at a junction between a spanning segment 4 and a tension-releasing portion 2, or on a tension-releasing portion 2.

In further embodiments, the steps of monitoring IOP and cutting an adjustable suture, if lower IOP is desired, are repeated until IOP in the subject is about 5 mm Hg to about 15 mm Hg. In some embodiments, cutting an adjustable suture lowers IOP in a subject by about 1 to about 5 mm Hg, about 5 mm Hg to about 10 mm Hg, about 10 mm Hg to about 20 mm Hg, about 10 mm Hg to about 30 mm Hg, about 1 mm Hg, about 3 mm Hg, about 5 mm Hg, about 7 mm Hg, about 10 mm Hg, about 15 mm Hg, about 20 mm Hg, about 25 mm Hg or about 30 mm Hg. In other embodiments, cutting an adjustable suture lowers IOP in a subject by about 1% to about 5%, about 5%, about 10%, about 15%, about 20%, about 25%, about 30%, about 40%, about 50%, about 60%, about 70% or about 80%.

There are further provided methods of incrementally lowering IOP post-operatively in a subject in need thereof, wherein the subject has undergone trabeculectomy surgery using an adjustable suture, comprising cutting the adjustable suture, e.g., by applying laser energy, at a selected site such that the adjustable suture is lengthened or loosened and IOP is lowered; and repeating this step until IOP in the subject is lowered to a selected level. In one embodiment, the selected IOP level is about 5 mm Hg to about 15 mm Hg. In another embodiment, glaucoma is treated in the subject.

Also provided is a method of performing trabeculectomy in a subject in need thereof, comprising performing trabeculectomy using an adjustable suture; monitoring the subject's IOP post-operatively; and, if lower IOP is desired, cutting the adjustable suture at a selected site such that the adjustable suture is lengthened or loosened and IOP is lowered. In an embodiment, glaucoma is treated in the subject.

In a particular embodiment, there is provided a method of treating glaucoma in a subject in need thereof, comprising: a) performing trabeculectomy using an adjustable suture; b) monitoring the subject's IOP post-operatively; c) if lower IOP is desired, cutting the adjustable suture at a selected site to release tension, such that the adjustable suture is lengthened or loosened and IOP is lowered; d) monitoring again the subject's IOP post-operatively; e) if lower IOP is desired, cutting the adjustable suture at a second selected site to release tension further, wherein the second selected site is on a spanning segment 4, at a junction between a spanning segment 4 and a tension-releasing portion 2, or on the tension-releasing portion 2; and f) optionally repeating steps d) and e) until IOP in the subject is lowered to a selected level or glaucoma is treated in the subject.

### BRIEF DESCRIPTION OF THE DRAWINGS

For a better understanding of the invention and to show more clearly how it may be carried into effect, reference will now be made by way of example to the accompanying drawings, which illustrate aspects and features according to embodiments of the present invention, and in which:

FIG. 1 shows a schematic representation of an embodiment of an adjustable suture of the invention having a “φ”-shape.

FIG. 2 illustrates a working principle of adjustable sutures, where the adjustable suture is as shown in (A); as shown in (B), the adjustable suture is cut at the spanning segment 4, e.g., using a surgical laser; and as shown in (C), cutting the spanning segment 4 causes the tension load to be redistributed to the tension-releasing portion 2. Thus cutting the spanning segment 4 loosens the adjustable suture.

FIG. 3 shows embodiments of adjustable sutures of the invention, where (A) shows an “8”-shaped adjustable suture; (B) shows a “$”-shaped adjustable suture; (C) shows a “B”-shaped adjustable suture; (D) shows a “D”-shaped adjustable suture; (E) shows a “§”-shaped adjustable suture; (F) shows a “Δ”-shaped adjustable suture; and (G), (H) and (I) show other embodiments of adjustable sutures.

FIG. 4 shows results from prototype testing for two prototyped sutures among the tested samples that carry the highest (labeled “1”) and lowest (labeled “2”) tension loads before failure occurs. The graph shows tension load (Force) applied along the suture (vertical axis) vs. axial ring deformation (Displacement; horizontal axis), with the spanning segment 4 of the adjustable suture 10 cut; “A” indicates a point where the tension load reaches the highest value, and “B” indicates where the suture fully failed due to separation of the core suture (the suture thread 1) from the ring (the tension-releasing portion 2).

FIG. 5 shows a schematic illustration of a through-cut hot embossing process. In (a), a nylon sheet and buffer layer are sandwiched by a rigid substrate plate and a mold insert with path patterns; in (b), the path patterns are pressed into the nylon with a thin residual layer left on the bottom of the blind grooves; in (c), the path patterns are pressed further down into the nylon and onto the buffer layer, ending in an indent with a depth greater than the thickness of the residual layer. The nylon residual layer is cut by the path pattern and sidewall of indents, and pressed into the bottom of the indentation. In (d), the insert is demolded and opened via grooves.

### DETAILED DESCRIPTION OF THE INVENTION

Described herein are adjustable sutures which are easy to use, reliable, and/or cost-effective. These adjustable sutures can be used in any surgical procedure where it is desirable to control surgical wound characteristics, e.g., to loosen sutures intra- or post-operatively, and where sutures are accessible, e.g., by laser or direct mechanical contact with scissors or a blade. For example, adjustable sutures can be used for trabeculectomy surgery for glaucoma. Also described herein are methods for making adjustable sutures, e.g., using micromanufacturing technology, and methods of use thereof.

For use in trabeculectomy, it is desirable for adjustable sutures to have a simple structure allowing for easy use and compatibility with existing suturing techniques. It is also advantageous to avoid requiring new skills to be learned by surgeons. Adjustable sutures should be safe and reliable and biocompatible to improve long term success. Ideally such adjustable sutures are capable of multiple incremental IOP lowering steps to achieve desired IOP control. Finally, production costs should be low so that sutures are economical and affordable. Adjustable sutures described herein provide some or all of these features. Adjustable sutures described herein provide eye surgeons the capability to adjust tension of a suture according to pressure in the eye. Adjustable sutures described herein can be lengthened or loosened incrementally and/or provide incremental IOP lowering.

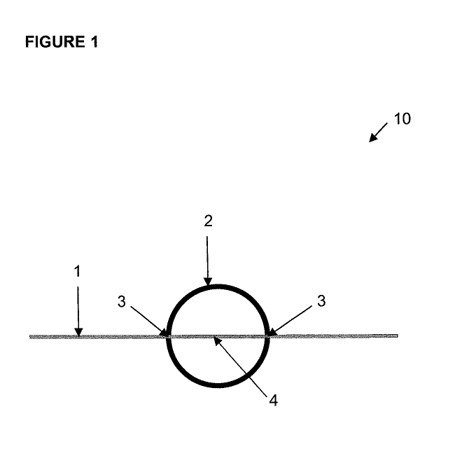

Adjustable sutures provided herein may integrate one or more tension-releasing portions or structures with existing sutures. In an embodiment, adjustable sutures comprise sutures that are currently used in surgery, e.g., eye surgery, and a simple add-on. In brief, a suture thread (1) passes through the center of an attached extendable structure (referred to herein as a tension-releasing portion 2) and takes the full tension. When necessary, a surgeon can cut the adjustable suture at a selected site, thereby engaging (i.e., transferring tension, in some instances making taut) the tension-releasing portion 2; consequently, the tension-releasing portion 2 will accept the tension, and relaxation of the adjustable suture 10 is provided relative to the pre-cut adjustable suture. (Throughout the Figures, numeral 10 refers generally to an adjustable suture.) A selected site for cutting an adjustable suture may be on a spanning segment 4 (a segment of the adjustable suture 10 which spans a tension-releasing portion 2 and joins the tension-releasing portion 2 at joining zones 3; see, for example, FIG. 1) or at a junction (i.e., a joining zone 3) between the spanning segment 4 and a tension-releasing portion 2. It should be understood that, when an adjustable suture 10 is cut for the first time, it should not be cut on a non-tension-bearing portion (i.e., tension-releasing portion 2); but rather on a tension-bearing segment such as a spanning segment 4. However subsequent cuts may be made on a spanning segment 4, at a junction between a spanning segment 4 and a tension-releasing portion 2, or on a tension-releasing portion 2. A site for cutting will be selected based on several factors such as the structure of an adjustable suture, previous cuts which have already been made, amount of relaxation, lengthening or loosening required, and so on. In embodiments where more than one tension-releasing portion 2 is present, each tension-releasing portion 2 is generally releasable independently, as long as the selected site of cutting is chosen appropriately. This allows step-wise or sequential lengthening or loosening of an adjustable suture by repeated cutting.

In some embodiments, a tension-releasing portion 2 is not aligned with the longitudinal axis of the adjustable suture 10, that is, it juts out from the longitudinal axis of the suture thread 1. In an embodiment, a surgeon can break (i.e., cut, lyse) a spanning segment 4 between adjacent joining zones 3, e.g., by using a laser; the tension-releasing portion 2 is then engaged and takes the tension, and relaxation of the adjustable suture is provided. In the case of trabeculectomy, the flap created in the patient's eye will be “loosened” and aqueous humor will flow out faster and pressure in the eye will drop accordingly.

It should be understood that any conventional means known in the art for cutting adjustable sutures may be used. Standard laser systems already widely used clinically may be used to break adjustable sutures. For example, in one embodiment an argon laser with a laser spot size of 50 μm is used. In other embodiments, adjustable sutures may be broken mechanically, for example using scissors or a blade. In the case where an adjustable suture of the invention is breakable by applying laser energy, the adjustable suture is also referred to as a “laser-adjustable suture”.

An embodiment of the adjustable suture of the invention is shown in FIG. 1. In this embodiment, the adjustable suture 10 comprises a suture thread 1 attached to a tension-releasing portion 2 (e.g., a ring in the embodiment shown in FIG. 1), with the suture thread 1 and the tension-releasing portion 2 joined together at two joining zones 3. The portion of the adjustable suture 10 which extends between the joining zones 3 is referred to as the spanning segment 4. In the embodiment shown in FIG. 1, the spanning segment 4 of the adjustable suture 10 spans the diameter of the tension-releasing portion 2 (i.e., the ring) forming a “φ” shaped device.

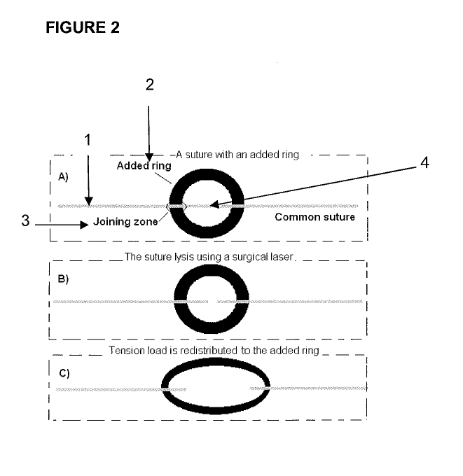

FIG. 2 illustrates schematically a working principle of an embodiment of the adjustable suture of the invention. When the spanning segment 4 is broken, tension is transferred to the tension-releasing portion 2 and the adjustable suture effectively lengthens. This serves to “loosen” the adjustable suture so that, in the case of trabeculectomy, flap closure tension in the eye is reduced and resistance to aqueous flow is lowered.

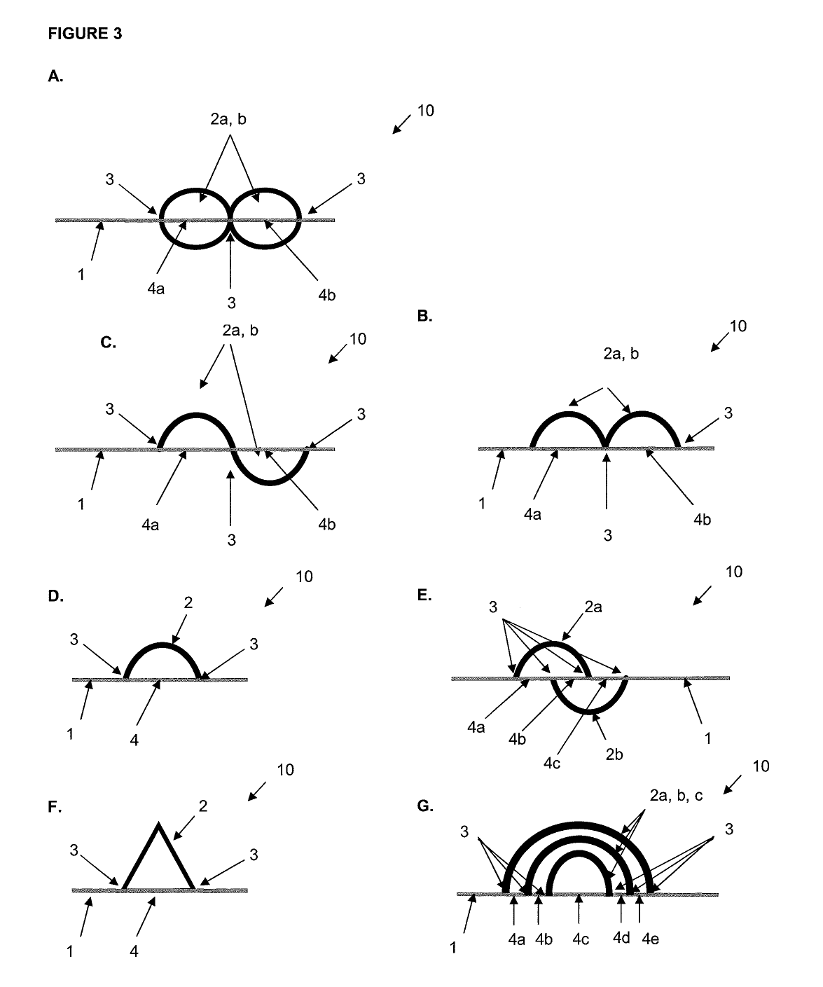

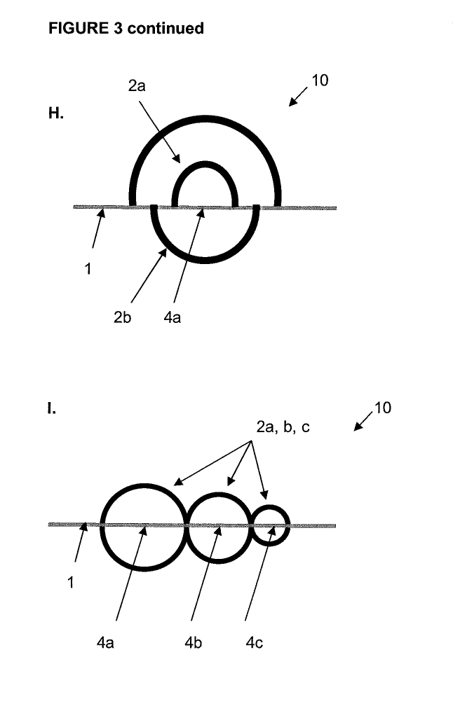

In another embodiment, an adjustable suture comprises a suture thread 1 attached to more than one tension-releasing portion 2 (as shown, for example, in FIGS. 3A, 3B, 3C, 3E, 3G, 3H and 3I; the more than one tension-releasing portions are referred to as 2a, 2b, 2c, etc.), with the suture 1 and the tension-releasing portions 2 joined together at joining zones 3. As there are more than one tension-releasing portions 2 (and more than two joining zones 3), there are also more than one spanning segments 4, each spanning segment 4 extending between a pair of adjacent joining zones 3 and each independently breakable (e.g., by laser or scissors). Multiple spanning segments 4 are referred to as 4a, 4b, 4c, etc. Multiple tension-releasing portions 2 may be located sequentially (as shown for example in FIGS. 3A, 3B, 3C, 3I); may be overlapping (as shown for example in FIG. 3E); or may be nested (as shown for example in FIGS. 3G, 3H), depending on the shape and configuration of the adjustable suture. In some embodiments, any one or more of the spanning segments 4a, 4b, 4c, etc. may be cut in order to loosen or lengthen an adjustable suture. In other embodiments, any one or more of the tension-releasing portions 2a, 2b, 2c, etc. may be cut to lengthen or loosen an adjustable suture, as long as at least one spanning segment 4 has previously been cut. In further embodiments, both at least one spanning segment 4 and at least one tension-releasing portion 2 may be cut to lengthen or loosen an adjustable suture.

In such embodiments, following cutting of the spanning segment 4, it is possible to further loosen the adjustable suture by cutting one or more of a plurality of tension-releasing portions 2. See, for example, the adjustable suture schematic of FIG. 3G. Thus, a first tension-releasing portion 2 effectively functions as a spanning segment 4 for a second tension-releasing portion 2, a second tension-releasing portion 2 functions as a spanning segment 4 for a third tension-releasing portion 2, etc. That is, when a plurality of tension-releasing portions are present, an adjustable suture is further lengthened or loosened, after a first tension-releasing portion 2 has already been cut, by cutting a second tension-releasing portion 2. This can be done by cutting an adjustable suture at a second selected site, subsequent to cutting a first selected site. These steps can be repeated as many times as permitted by the structure and accessibility of an adjustable suture until an adjustable suture has a desired tightness or looseness. When cutting an adjustable suture for a second, third, or fourth, etc., time, the adjustable suture may be cut on a spanning segment 4, at a junction between a spanning segment 4 and a tension-releasing portion 2, or on a tension-releasing portion 2.

In one embodiment, adjustable sutures comprise a nylon ring-shaped tension-releasing portion 2 and a standard 10-0 nylon suture thread 1. In another embodiment, adjustable sutures comprise a nylon ring-shaped tension-releasing portion 2, a standard 6-0 nylon suture thread 1 and nylon adhesive, such as cyanoacrylate. It should be understood that sutures of the complete range of commercially available calibers (suture thread diameters) and materials may be used in adjustable sutures of the invention. In yet another embodiment, Silastic® (silicone rubber; Dow Corning Corporation, Missouri, U.S.A.) is used to make a tension-releasing portion. Silastic® is transparent and rarely absorbs laser energy. This makes the device resistant to laser damage during post-surgical laser treatment.

It will be appreciated by a person of ordinary skill in the art that any material which is biocompatible and can be attached to a suture thread 1 may be used to make tension-releasing portions 2, and is encompassed herein. For example, tension-releasing portions may be made of nylon 6, nylon 6.6, silicone elastic polymer, Silastic®, silicone rubber or other materials which are used in standard sutures, or combinations thereof. In one embodiment, the material used to make a tension-releasing portion does not absorb laser energy. In an embodiment, the material used to make a tension-releasing portion is not absorbable (i.e., not absorbable means does not dissolve in a living system, is not biodegradable). In another embodiment, a tension-releasing portion 2 is made of a material which is deformable, extendable or flexible. In yet another embodiment, a tension-releasing portion 2 is made of a material which is ductile or elastic.

The size and shape of an adjustable suture will vary depending on the particular requirements of the surgery in question, such as the surgery being performed, the tissue being sutured, how the adjustable suture is to be broken, the laser to be used, etc. It is expected that different adjustable suture designs may provide different relaxation features. The skilled artisan will select an adjustable suture design for use based on the particular requirements of the surgery in question. The size and shape of a tension-releasing portion, e.g., a ring, rectangle, etc., will also vary depending on how an adjustable suture is to be broken, the laser to be used, the size of the suture thread 1, the surgery being performed, etc. Non-limiting examples of other embodiments of adjustable sutures of the invention, such as “D”-, “$”-, “Δ”-, “B”-, “8”- and “§”-shaped sutures, are shown in FIG. 3.

In one embodiment, the internal diameter of a tension-releasing portion 2, e.g., a ring, varies from about 0.25 mm to about 1.5 mm; in a particular embodiment, the internal diameter of a tension-releasing portion 2 is about 1.0 mm to about 1.5 mm, about 1 mm, about 1.25 mm, or about 1.5 mm. In one embodiment, the thickness of a tension-releasing portion 2 is about 0.25 mm, the inner diameter (ID) of the tension-releasing portion 2 is about 1 mm, and the outer diameter (OD) of the tension-releasing portion 2 is about 1.5.

Many other sizes and shapes are possible and are encompassed by the invention. It is contemplated that sutures of all possible calibers, used in any type of surgery, may be used in adjustable sutures of the invention. “Caliber” refers to the diameter of the suture thread. The size of a suture or the material used to make a tension-releasing portion will vary. For example, larger diameters would be used when greater suture strength or tension is required (as might be expected for example in general surgery, orthopedic surgery, neurological surgery, cardiac surgery, and other types of surgery). In general, a tension-releasing portion 2, e.g., a ring, should be sized appropriately to allow the surgeon to handle an adjustable suture efficiently and precisely while being small enough for implantation into the desired tissue, e.g., the eye. Without wishing to be limited by example, sutures of size 2-0, 3-0, 4-0, 5-0, 6-0, 7-0, 8-0, 9-0, 10-0 and 11-0 may be used.

It is contemplated that any size and type of suture known in the art can be used for adjustable sutures of the invention. In one embodiment, a suture thread 1 and/or tension-releasing portion 2 are made of a deformable material or a flexible material. In another embodiment, a suture thread 1 and/or tension-releasing portion 2 are made of a non-deformable material or an inflexible material. In another embodiment, a suture thread 1 and/or tension-releasing portion 2 are made of silicone elastomer (preferably medical grade), such as silicone elastomer from NuSil Silicone Technology (California, U.S.A.). In yet another embodiment, a suture 1 and/or tension-releasing portion 2 are made of nylon, silk, polyester, polypropylene or other known suture materials, or a combination thereof. In another embodiment, a suture 1 and/or tension-releasing portion 2 are made of nylon 6 or nylon 6.6 and nylon adhesive. In one embodiment, a suture thread 1 and/or tension-releasing portion 2 are made of 2-0, 3-0, 4-0, 5-0, 6-0, 7-0, 8-0, 9-0, 10-0 or 11-0 nylon.

Many types of sutures are known in the art and may be used in adjustable sutures of the invention. Non-limiting examples of suture types which may be used include: monofilament sutures such as polypropylene sutures, catgut, nylon, PVDF, stainless steel, poliglecaprone and polydioxanone sutures; multifilament or braided sutures such as PGA sutures, polyglactin 910, silk and polyester sutures; absorbable sutures such as polyglycolic acid sutures, polyglactin 910, catgut, poliglecaprone 25 and polydioxanone sutures; non-absorbable sutures such as polypropylene sutures, nylon (poylamide), polyester, PVDF, silk and stainless steel sutures; synthetic sutures; and natural sutures such as silk and catgut sutures, and combinations thereof.

A suture thread 1 and tension-releasing portion 2 may be made of the same or different material.

In certain embodiments, such as adjustable sutures shown in FIGS. 3A, 3B, 3C, 3E, 3G, 3H and 3I, an adjustable suture has more than one tension-releasing portion 2 and consequently more than two joining zones 3. In some embodiments, an adjustable suture has more than one spanning segment 4, each spanning segment 4 extending between a pair of adjacent joining zones 3. Each spanning segment 4 is independently breakable, e.g., by laser, allowing adjustable sutures to be incrementally lengthened or loosened sequentially, as desired. For example, a first spanning segment 4a extending between a first pair of adjacent joining zones 3 can be broken. If further lengthening or loosening of an adjustable suture is desired, e.g., further lowering of a subject's IOP is desired, then optionally a second spanning segment 4b extending between a second pair of adjacent joining zones is broken; and so on. This procedure can be repeated as many times as possible based on the structure of the adjustable suture used (i.e., the number of tension-releasing portions 2 or their configuration) and the accessibility of the adjustable suture, allowing stepwise, incremental lengthening or loosening of the adjustable suture.

In some embodiments, it may be desirable to break one or more of the tension-releasing portions 2, in addition to breaking a spanning segment 4. For example, when multiple, sequential, overlapping, and/or nested, tension-releasing portions 2 are present, incremental loosening of an adjustable suture can be achieved by breaking one or more of the tension-releasing portions 2, subsequent to initial breaking of at least one spanning segment 4. As an example, see FIG. 3H, where an embodiment with multiple nested tension-releasing portions 2 is shown. In this case, a spanning segment 4a may be optionally broken first; subsequently, if further loosening of the adjustable suture is required, then the tension-releasing portion 2a may optionally be broken; subsequently, the tension-releasing portion 2b may optionally be broken. Thus, in some embodiments tension-releasing portions 2 are breakable, e.g., by applying laser energy or mechanically. In some methods provided herein, tension-releasing portions 2 of adjustable sutures are optionally broken, subsequent to a first step of breaking a spanning segment 4.

In an embodiment, adjustable sutures are fabricated by adding a tension-releasing portion 2 to an existing suture thread 1. In an embodiment, integration of attached extendable structures (“tension-releasing portions 2”) with existing sutures is accomplished using a micromoulding process. For example, a high-speed micromilling machine can be used to manufacture micro moulds. In another embodiment, a suture thread 1 and tension-releasing portion 2 are fabricated together in one piece. For example, to fabricate the suture 1 and the tension-releasing portion 2 together in one piece, direct printing can be used. In direct printing methods, a micronozzle is used to directly print a thin layer of diamine in a groove patterned on a metal mould, then another micronozzle is used to print a thin diacid layer on top of the diamine layer. A reaction of diamine and diacid makes substrates nylon 6.6 and water. The substrates will then be placed on a hotplate with an elevated temperature to remove water and to accelerate the polymerization process. Thus, depending on methods used for fabrication, an adjustable suture may have a tension-releasing portion 2 integrated with the suture thread 1 in one piece, or may have a tension-releasing portion 2 attached or added to the suture thread 1. In the latter case, a tension-releasing portion 2 may or may not be fixed in place on the suture thread 1.

There are also provided herein surgical methods for use of adjustable sutures described herein. For example, there is provided herein a method of performing trabeculectomy using adjustable sutures of the invention. There is further provided a method of incrementally lowering IOP post-operatively in a subject in need thereof, wherein the subject has undergone trabeculectomy surgery using adjustable sutures described herein, comprising breaking a spanning segment 4 of the adjustable suture 10, such that the adjustable suture is loosened and IOP is lowered. If multiple spanning segments 4 are present, then spanning segments 4 may be broken sequentially, as desired. In some embodiments, a spanning segment 4 of the adjustable suture 10 is broken using a laser (i.e., applying laser energy). In other embodiments, a spanning segment 4 is broken mechanically, e.g., using scissors or a blade. In further embodiments, more than one spanning segments 4 are broken optionally to lower IOP to a selected level. In still further embodiments, tension-releasing portions 2 are broken optionally to lower IOP to a selected level, as long as at least one spanning segment 4 has first been broken in the adjustable suture.

In an embodiment, there is provided a method of treating glaucoma comprising performing trabeculectomy in a subject using adjustable sutures described herein; monitoring the subject's IOP post-operatively to determine if it is desirable to lower the subject's IOP; and, if it is desired to lower IOP, breaking a spanning segment 4 of the adjustable suture 10, e.g., using a laser. If adjustable sutures have more than one tension-releasing portion 2, then a spanning segment 4 is first broken (e.g., spanning segment 4a). Subsequently, if it is desired to lower IOP further, a second spanning segment 4 is optionally broken (e.g., spanning segment 4b) extending between a second pair of adjacent joining zones 3; these steps are repeated as many times as desired and as possible depending on the structure and accessibility of the adjustable suture (e.g., if there are three spanning segments extending between three pairs of adjacent joining zones, then IOP can be lowered step-wise three times, by sequentially cutting each of the three spanning segments 4a, 4b and 4c extending between the three pairs of adjacent joining zones). In further embodiments, tension-releasing portions 2 are broken optionally to lower IOP to a selected level, as long as a spanning segment 4 has first been broken in the adjustable suture.

In some embodiments, methods provided herein comprise breaking one or more tension-releasing portions 2. In such embodiments, a spanning segment 4 of an adjustable suture is first broken. Subsequently, if it is desired to loosen the adjustable suture further, a second spanning segment (e.g., spanning segment 4b) extending between a second pair of adjacent joining zones 3 is broken, or a tension-releasing portion 2 is broken. At each subsequent loosening of an adjustable suture, either another spanning segment 4 or another tension-releasing portion 2 is broken, depending on the shape or configuration of the adjustable suture, the amount of loosening desired, accessibility, etc.

The amount of IOP lowering achieved by breaking an adjustable suture (e.g., by breaking a spanning segment 4 or a tension-releasing portion 2) will vary depending on the adjustable suture used. In one embodiment, IOP is lowered by about 1 to about 5 mm Hg, about 5 mm Hg to about 10 mm Hg, about 10 mm Hg to about 20 mm Hg, about 10 mm Hg to about 30 mm Hg, about 1 mm Hg, about 3 mm Hg, about 5 mm Hg, about 7 mm Hg, about 10 mm Hg, about 15 mm Hg, about 20 mm Hg, about 25 mm Hg or about 30 mm Hg, each time an adjustable suture is broken, e.g., each time a spanning segment 4 or a tension-releasing portion 2 is broken. In another embodiment, IOP is lowered by about 1% to about 5%, about 5%, about 10%, about 15%, about 20%, about 25%, about 30%, about 40%, about 50%, about 60%, about 70% or about 80% each time an adjustable suture is broken, e.g., each time a spanning segment 4 or a tension-releasing portion 2 is broken.

A person of ordinary skill in the art will determine when it is desirable to lower IOP in a subject based on professional expertise and experience. Without wishing to be limited by example, an IOP of about 5 mm Hg to about 15 mm Hg is typically desirable. IOP can be measured using standard techniques known in the art.

“Sufficiently lowering” IOP, or lowering IOP “to a selected level,” “as desired” or to a “desired level,” refers to achieving a level of IOP which is expected by the artisan to stabilize glaucoma (e.g., prevent further vision loss). In an embodiment, IOP is sufficiently lowered or is lowered to a selected level if glaucoma is lessened, mitigated, alleviated or eliminated. A selected or sufficient level of IOP will depend on many factors such as the subject, the disease condition, the surgical outcome, etc., and is determined by the skilled artisan based on professional expertise and experience. An artisan will determine, for example, whether an additional spanning segment 4 or tension-releasing portion 2 should be broken in order to provide the best possible clinical outcome for the subject.

In another embodiment, there is provided a method of loosening a suture intra- or post-operatively in a subject, wherein the subject undergoes or has undergone surgery using adjustable sutures described herein, comprising breaking a spanning segment 4 of the adjustable suture 10, or a tension-releasing portion 2, e.g., using a laser or scissors, such that the adjustable suture is loosened. For example, adjustable sutures may be used for other eye surgeries such as cataract surgery. More generally, it is contemplated that adjustable sutures may be used in any procedure where a suture is tied and may subsequently need to be precisely loosened. Adjustable sutures may be used, for example, in general surgery, orthopedic surgery, neurological surgery, cardiac surgery, and other types of surgery. For some surgeries, loosening may occur intra-operatively, e.g., during cardiac valve surgery, during neurosurgery, etc.

In some embodiments, it is possible to visually distinguish different portions of an adjustable suture from one another. For example, a first colour may be used to show which portion is the spanning segment 4, a different colour may indicate the tension-releasing portion 2, and/or a different colour may distinguish the regions of the suture thread 1 flanking the spanning segment 4. In some embodiments, texture or another visual and/or tactile characteristic may be used to distinguish different areas of an adjustable suture.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Example 1

### Manufacture of Adjustable Sutures

Adjustable sutures comprising a ring-shaped tension-releasing portion 2 and a standard 10-0 nylon suture thread 1 were fabricated. Suture thread 1 spans the diameter of a ring-shaped tension-releasing portion 2, wherein the portion of the suture thread that spans the ring is referred to as spanning segment 4, forming a “φ” shaped device. As argon lasers with a laser spot size of 50 μm are widely available in clinical settings, this size was used to determine suitable ring sizes. A ring-shaped tension-releasing portion 2 was made using silicone elastomer (medical grade) from NuSil Silicone Technology (California, U.S.A.). Adjustable sutures with a ring size of about 0.25 mm in thickness, an outer diameter (OD) of about 1.5 mm and an inner diameter (ID) of about 1 mm were prepared.

Micromolding was used to fabricate the adjustable sutures. A Microlution™ high speed micromilling machine was used to develop micromolds for the ring and suture. A drop of liquid silicone elastomer mixed with its curing agent was deposited into the mold, which sits on a spin coater to remove the excess silicone. The mold was placed on a hot plate at 150° C. for 10 minutes, then cooled to room temperature. The nylon suture was thus joined with the silicone ring tightly and released from the micromold. In order to ensure that the ring and the suture are properly joined, care must be taken to design the mold delicately and to machine it properly.

### Example 2

### Physical Testing of Adjustable Sutures

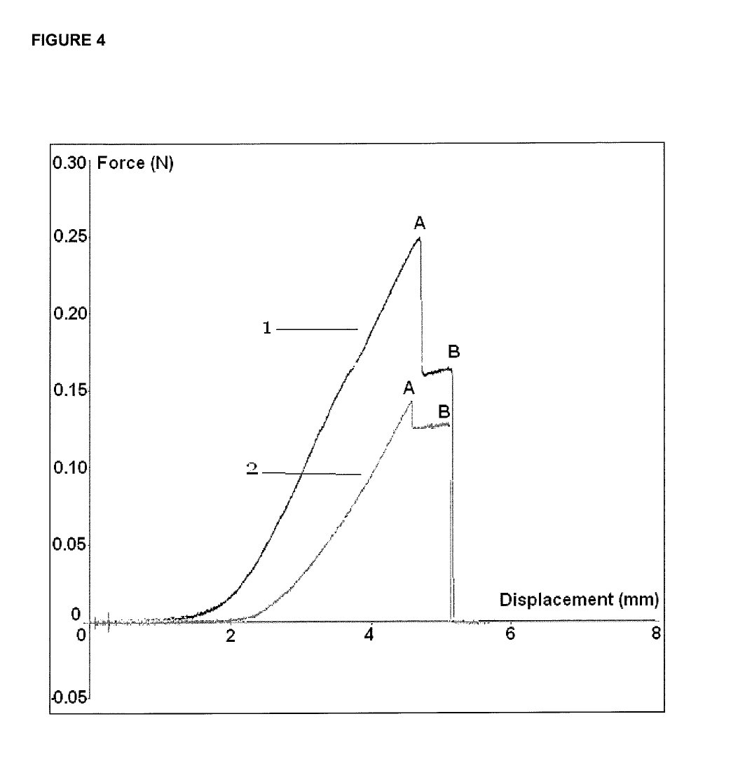

To examine the bonding strength of the joining zone, a spanning segment 4 of a suture was cut and tensile testing was conducted for a set of 12 identical φ-shaped adjustable suture prototypes with a ring size of about 0.25 mm in thickness, an outer diameter (OD) of about 1.5 mm and an inner diameter (ID) of about 1 mm. Suture prototypes consisted of a 10-0 nylon core suture and a silicone ring. The suture 1 slipped out of the joining zone 3 and separated from the ring (the tension-releasing portion 2) (from A to B in FIG. 4) at a mean tension of 0.189±0.032 N. This force caused deformation of approximately 5 mm before the suture thread 1 slipped out of the joining zone 3 and separated from the ring (i.e., before the suture “pulled-out”). Without wishing to be limited by theory, in glaucoma applications an expected deformation (relaxation) that will be needed to induce a required reduction in resistance to aqueous outflow is expected to be below 1 mm.

In summary, adjustable sutures included a silicone rubber ring-shaped tension-releasing portion 2 that was capable of elongating about 4 mm under a maximum pulling load of about 0.18 N before failure. Elongation of 4 mm is more than adequate for most clinical, surgical purposes. These results indicate structural reliability of adjustable sutures since the actual tension load is expected to be far below the maximum load applied during this mechanical testing. The actual tightening load on a surgically tied suture is generally well below the critical threshold load where “pull out” (separation of suture thread 1 from ring or tension-releasing portion 2) occurs.

In other studies, to prove the safety of adjustable sutures under tension, experiments were conducted to determine the maximum “pull-out” load. Adjustable sutures were made having different ratios of silicone base to cure agent. Such differences lead to differences in elongation and tensile force. Experiments were conducted using different silastic ratios, different cure temperatures, and different cure durations. Texture analysis was used to measure maximum “pull-out” force as well as load-displacement curves.

### Example 3

### Ex Vivo Laboratory Simulation Testing of Adjustable Sutures

Trabeculectomy surgery was performed on human cadaver eyes with prototype adjustable sutures. Eyes were perfused with an infusion pump and pressure within the eye was monitored. Changes in pressure and outflow facility from baseline were measured while sequential suture adjustments were carried out. Specifically, flow rate was first adjusted to obtain steady-state conditions at a preset initial IOP, and three successive modifications to the adjustable suture were tested: 1) the central suture spanning the ring was cut (i.e., the spanning segment 4 spanning the ring was cut); 2) one side of the device was cut to investigate the effect of increasing device deformability; and 3) the 10-0 suture (suture 1) was completely cut.

Results were obtained from four eyes. Perfusion rates of 0.6 to 4.8 ml/hr achieved baseline pressures that ranged from 23.3 to 28.7 mm Hg. Cutting the central part of the suture spanning the ring (the spanning segment 4) produced a median IOP decrease of 36.8%. Cutting one side of the device further lowered IOP by a median of 5.5%. Completely cutting the adjustable suture resulted in a severe IOP drop to a level ranging from 3.1 to 5.9 mm Hg. These results indicate that adjustable sutures can be used to lower IOP incrementally in perfused human eyes, by increasing facility of aqueous humor outflow in a regulated way.

### Example 4

### Fabrication Techniques

It is desirable to provide simple and cost-effective manufacturing and fabrication methods for adjustable sutures. Two fabrication technologies are presented: hot embossing and microwelding. Both techniques require micromolds which are manufactured using a high-speed micromilling machine.

### i) Making Micromolds Using a High Speed Micromilling Machine:

Here, a high performance Microlution™ (S362) micromilling machine, with spindle speed up to 100,000 rpm allowing customized free-form manufacturing, was used to develop micromolds. Brass was used to make molds since it possesses good thermal conductivity and is easy to cut. A path pattern, which defines the geometry of the added load redistribution features, was formed by grooves in the base. For the microwelding fabrication approach, aluminum will be used to make molds and the base will have precise temperature control allowing easy release from the base.

### Ii) Making Adjustable Sutures Using Hot Embossing:

The hot embossing process is performed in HEX01 (JENOPTIK, Germany). There are two hot plates in this machine: a bottom stationary and a top movable hot plate. The maximum force and temperature are 50 kN and 320° C., respectively. A rotary pump is connected to an embossing chamber to provide vacuum lower than 0.1 mbar. Two methods are used: hot embossing nylon 6 sheets and hot embossing existing nylon sutures.

First, nylon 6 sheets (also called thin films) with thicknesses of 25 μm and 35 μm from Goodfellow™ are used. A 4-inch silicon wafer is used as workpiece holder attached to the bottom stationary hot plate of the hot embossing machine. On top of the wafer, a layer of SU-8 3050 photoresist (MicroChem, USA) is spin-coated, soft-baked at 95° C. for 15-30 min, and exposed to UV light at a dose of 250-300 mJ/cm². Then it is post-baked at 95° C. for 5 min. The nylon sheet is placed on top of the cured SU-8 layer, which is used as a cushion or buffer layer (hardness of ˜400 MPa) (AlHalhouli, A. T., Microelectronic Engineering, 2008, 85:942-4; Zhong, Z. W., Materials Science, Poland, 2007, 25(1)). This has far lower than the minimum reported hardness of single crystal silicon (>5.1 GPa) (Bhushana, B. and Lia, X., J. Materials Research, 12(1):54-63).

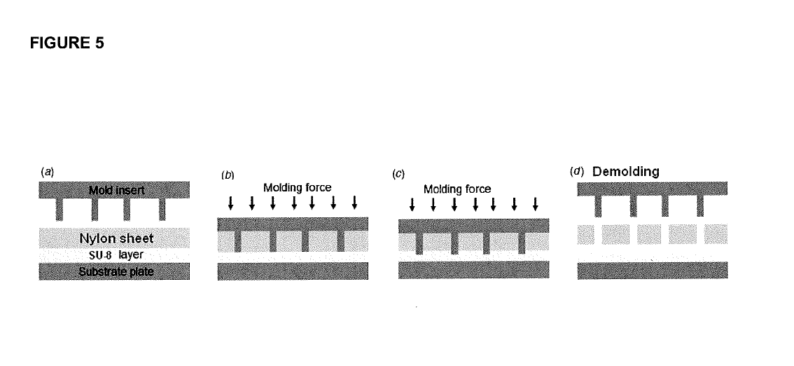

Instead of using the most common indentation process to make microfluidics channels, a cut-through fabrication process is used. This consists of two stages: forming blind micro grooves by hot embossing, and removing a residual layer by indentation, as shown in FIG. 5. Compared to conventional experimental setups, an additional buffer layer is added which has lower hardness than the mold insert, but has much greater hardness than nylon in the molding stage. After they are heated up to a molding temperature, path patterns on the mold insert—which create the precise shape desired (e.g., “φ”)—are pressed further into the nylon to form blind grooves with a thin residual layer left on the bottom.

Next they are pressed into the buffer layer as an indentation with a depth greater than the thickness of the residual layer. The residual layer is removed by the pins and the sidewall of indents. Therefore the grooves are opened in this continuous operation during a single cycle of a conventional hot embossing process. A new suture is shaped from the nylon film and released from the mold. An annealing procedure then takes place to allow the new adjustable suture device to self-absorb any burrs on the edges. The molding and demolding temperatures and forces, the depth of the path patterns, as well as the thickness of the buffer layer are optimized throughout.

A second hot embossing approach using, e.g., commercially available nylon 6 monofilament sutures (size 3-0) with diameter of approximate 200 μm as starting material, can also be used. A suture is placed on a silicon wafer attached to the bottom hot plate of the hot embossing machine and heated up to 50 to 90° C. (above nylon 6 glass transition temperature (47° C.) but lower than its melting point (220° C.)). Then a silicon wafer attached to the top hot plate of the machine is heated to the same temperature and pressure is applied to the suture for several minutes. The circular suture with cross-section of ˜200 μm in diameter is flattened to a rectangular shape with cross-section of ˜25×1500 μm (t×w). Then the flattened suture undergoes the same process described above. As such, new sutures with cross-sections of ˜25 μm (approximate diameter of 10-0 nylon sutures used in glaucoma surgery) are obtained from the original monofilament sutures. The molding and demolding temperatures, forces, the depth of the path patterns, as well as the thickness of the buffer layer are refined and optimized.

While these processes change the geometry of the materials, the chemical chain structure of the nylon remains untouched. Therefore, characteristics of the new adjustable sutures will be the same as the original materials. Either of the above two processes can be incorporated into industrial nylon production lines.

iii) Making Adjustable Sutures Using Microwelding:

Nylon can be welded in many different ways using laser, ultrasonic, thermal and chemical bonding (e.g., glue welding) techniques. For simplicity and durability, resorcinol glue (using 95% ethanol [CH₃CH₂OH] and resorcin [C₆H₄(OH)₂] mixed in 1:1 weight ratio) are utilized to weld nylon suture components together. Brass and/or aluminum micromolds are manufactured using micromilling machines, with geometries of adjustable sutures grooved in the molds. At the places where the added features (i.e., tension-releasing portions 2) cross over the central suture component (i.e., suture 1), a small semi-spherical blind pocket 50 μm in diameter with 10 μm radius fillets is created to retain chemical glue for welding. A nylon 6 monofilament surgical suture (size 10-0) with diameter of ˜25 μm is used for both the central spanning segment (4) and added load-redistribution features (tension-releasing portions 2).

The central suture is placed and secured in a groove and another identical suture is inserted into the grooves that define the geometry of the added features. Using a micro-nozzle, a drop of resorcinol glue is dispensed into the pockets where the central spanning segment (4) and added features (tension-releasing portions 2) meet. Then the mold is placed in a pressure chamber with air pressure of 80 kPsi and temperature of about 60 to about 80° C. The pressurized chamber provides a uniform pressure for the joint that ensures quality welding between sutures. In order to ensure repeatability of the fabrication process, a 3-axis Newport motion stage (computer controllable using LabVIEW) and a pressure driven micro dispenser are used to accurately place a precise amount of adhesive.

Using ultrasonic welding, a looped suture is welded at the junctions. A looped suture is placed on a heated surface with controlled temperature. An ultrasound welding probe passes energy to the suture. Most of the energy is absorbed where the loop overlaps. A looped suture is further heated to its melting temperature so that overlapps (junctions) join together.

Similarly, nylon suture can be welded by using laser or thermal welding, which provides local thermal energy to a junction and heats the suture to a certain temperature so that any overlapps (junctions) are bonded.

### iv) Other Fabrication Techniques:

Adjustable sutures can be made using riveting. Clamps (e.g., “+” and “O”-shaped finger-holders) may be used to locate and fix suture junctions. When sutures are placed into finger-holders, junctions can be closed and joined tightly.

Chemical welding techniques can also be used to prepare adjustable sutures. Biocompatible chemical adhesives are suitable for use as adhesives at junctions of adjustable sutures. Such materials include cyanoacrylates, light curing, epoxides and urethanes. Adjustable suture junctions can also be joined by clamps, knots, glue (e.g., cyanoacrylate, Loctite 4311 Flashcure (available from Henkel)), ultraviolet (UV) welding, solvent bonding, hot melt, and any other such techniques that effectively joins abutting or overlapping suture thread.

### Example 5

### Modeling and Optimization of Load Redistribution Features

To optimize relaxation and load redistribution features of adjustable sutures, stress and strain distribution in adjustable sutures are investigated. Tightness (tension load) of an adjustable suture for trabeculectomy varies depending on the surgeon's judgment during the operation. Estimated suture tension load is used in a 3D finite element model of adjustable sutures using COMSOL Multiphysics™. Through such modeling, layout and dimensions of load redistribution features, as well as geometry of the joining zone 3 (see FIG. 3) can be optimized. Several other designs (“D”, “$”, “□”, “B”, “8” and “§”, etc.; see FIG. 3), which provide different relaxation features, are similarly simulated and optimized.

### Example 6

### Mechanical Property and Fabrication Testing

Mechanical testing of adjustable sutures to optimize load redistribution structure for use in surgery, e.g., glaucoma surgery, is conducted. Manufacturing quality of micromolds, including surface roughness and size accuracy is examined using a topography measurement system (TMS Polytec™, Germany) and microscopes in our laboratory. Manufacturing parameters, including plunge rate, feed rate, spindle speed, and cutting depth are tuned to produce the best quality micro molds. Second, surface quality and geometry of adjustable sutures are examined, and parameters for hot embossing and micromolding processes, such as, molding and demolding temperatures, molding force, etc. are determined. Mechanical properties of adjustable sutures under tensile loads is tested using Texture Analyzer TA.XTpIus™ (Texture Technologies). The testing is conducted on adjustable sutures before and after the central ring-spanning segment 4 of the adjustable suture 10 is cut (i.e., before and after the spanning segment 4 is cut).

### Example 7

### Ex-Vivo Testing

Ex-vivo assessment of adjustable suture pressure control capabilities using cadaveric eyes is conducted.

In an embodiment, adjustable sutures are designed to provide flexibility to adjust aqueous outflow via relaxation of adjustable sutures in a predictable, incremental manner. To test this, a simulated inflow-outflow testing system is created using cadaveric eyes. Briefly, a syringe pump is used to deliver fluid at a rate of ˜3 μL/min to a cadaver eye in which a trabeculectomy has been performed using adjustable sutures. The adjustable sutures are over-tightened to simulate the clinical situation in which a suture would need to be “adjusted” to lower IOP. Then the central spanning segment (i.e., the spanning segment 4) is cut and change in IOP is measured. IOP is continuously monitored using pressure catheters and a PowerLab system (ADInstruments™). Once initial steady state pressures are achieved, another portion of the adjustable suture is cut (e.g., another spanning segment 4 or a tension-releasing portion 2 is cut) and pressure responses are recorded. It is determined whether adjustable sutures can consistently achieve desired IOP (e.g., 8-12 mm Hg) with physiologic fluid inflow (e.g., 2.5-3.0 μL/m in).

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

The contents of all documents and references cited herein are hereby incorporated by reference in their entirety.

1. An adjustable suture comprising:
a suture thread 1 and a tension-releasing portion 2, wherein the tension-releasing portion 2 is adapted to accept tension when the adjustable suture is cut at a selected site.

2. The adjustable suture of claim 1, wherein the adjustable suture comprises a portion which is a spanning segment 4, and the selected site is on the spanning segment 4 of the adjustable suture or at a junction between the spanning segment 4 and the tension-releasing portion 2.

3. The adjustable suture of claim 1, wherein the selected site is not on the tension-releasing portion 2.

4.-5. (canceled)

6. The adjustable suture of claim 1, wherein the tension-releasing portion 2 is integrated with the suture thread 1.

7. The adjustable suture of claim 1, wherein the adjustable suture is lengthened or loosened when the adjustable suture is cut at the selected site.

8. The adjustable suture of claim 1, wherein the adjustable suture comprises more than one tension-releasing portion 2.

9. The adjustable suture of claim 8, wherein each tension-releasing portion 2 is independently releasable.

10.-13. (canceled)

14. The adjustable suture of claim 1, wherein the adjustable suture is cut by breaking the adjustable suture mechanically or by applying laser energy to the adjustable suture.

15. The adjustable suture of claim 1, wherein the tension-releasing portion 2 is substantially ring-shaped, rectangular-shaped or triangular-shaped.

16. The adjustable suture of claim 1, wherein the adjustable suture is substantially φ-, D-, Δ-, B-, 8- or §-shaped.

17. The adjustable suture of claim 1, wherein the adjustable suture comprises nylon, silicone elastic polymer, Silastic®, silicone rubber, silk, polyester, polypropylene and/or other biocompatible suture materials.

18.-20. (canceled)

21. The adjustable suture of claim 1, wherein the suture thread 1 and the tension-releasing portion 2 are made of the same material.

22.-24. (canceled)

25. The adjustable suture of claim 1, wherein the tension-releasing portion 2 is made of deformable or flexible material.

26.-47. (canceled)

48. A method of treating glaucoma in a subject in need thereof, comprising:
a) performing trabeculectomy using the adjustable suture defined in claim 2;
b) monitoring the subject's intraocular pressure (IOP) post-operatively; and
c) if lower IOP is desired, breaking at least one spanning segment 4, such that the adjustable suture is loosened and IOP is lowered.

49. (canceled)

50. The method of claim 48, further comprising repeating steps b) and c) until the IOP in the subject is lowered to a selected level.

51.-52. (canceled)

53. The method of claim 48, wherein steps b) and c) are repeated until the IOP in the subject is about 5 mm Hg to about 15 mm Hg.

54.-68. (canceled)

69. A laser-adjustable suture comprising:
a suture thread 1 attached to one or more tension-releasing portion(s) 2, wherein the suture thread 1 and the tension-releasing portion 2 are joined together at two or more joining zones 3, and a spanning segment 4 extends between the joining zones 3;
wherein the laser-adjustable suture is breakable by applying laser energy; and
wherein, when more than one tension-releasing portion is present, more than one spanning segment 4 is present, each of said spanning segments 4 extending between a pair of adjacent joining zones 3 and independently breakable by applying laser energy.

70.-90. (canceled)

91. A method of making the adjustable suture of claim 1, comprising: using a micromolding, hot embossing, ultrasonic welding, riveting, knotting, clamping, gluing, or chemical welding technique or any combination thereof to form an adjustable suture.

92. The method of claim 91, wherein the adjustable suture comprises spin coated nylon, silicone elastic polymer, Silastic®, silicone rubber, silk, polyester, polypropylene, biocompatible suture materials, or any combination thereof.

93. The adjustable suture of claim 2, wherein different portions of the adjustable suture are visually distinguishable from one another.

94. (canceled)
